(19) United States
(12) Patent Application Publication
(10) Pub. No.: US 2017/0301032 A1
(43) Pub. Date: Oct. 19, 2017

(54) PATIENT DATA TRIGGERED SYSTEM FOR RISK TRANSFER LINKED TO PROLONGING INDEPENDENT LIVING BY ELDERLY ILLNESS OCCURRENCE AND CORRESPONDING METHOD THEREOF

(71) Applicant: Swiss Reinsurance Company Ltd., Zurich (CH)

(72) Inventors: John Turner, Waedenswil (CH); Melissa Leitner, Waedenswil (CH); Alan Martin, Ivychurch (GB); Alan Dott, Ipswich (GB); Poppy Girdlestone, Chemsford (GB)

(73) Assignee: Swiss Reinsurance Company Ltd., Zurich (CH)

(21) Appl. No.: 15/637,454

(22) Filed: Jun. 29, 2017

Related U.S. Application Data
(63) Continuation of application No. PCT/EP2015/073363, filed on Oct. 9, 2015.

(51) Int. Cl. G06Q 40/08; G06Q 50/22; G06Q 50/24

(52) U.S. Cl. CPC G06Q 40/08; G06Q 50/22; G06Q 50/24

(57) ABSTRACT

A resource pooling system and method for multi-pillar triggered risk transfer associated with prolonged independent living under elderly illness occurrence by providing dynamic self-sufficient risk protection for a variable number of risk exposure components by the resource pooling system. The risk exposure components are connected to the resource pooling system by a plurality of payment receiving devices configured to receive and store payments from the risk exposure components for the pooling of their risks and resources. The resource pooling system includes a filter device configured to capture age-related parameters of risk exposure components and filter risk exposure components associated with an age-related parameter greater than a predefined age-threshold value by the predefined age-threshold value.

### CROSS REFERENCE TO RELATED APPLICATION

This application is a continuation of PCT International Application No. PCT/EP2015/073363, tiled on Oct. 9, 2015, the entire contents of which are incorporated herein by reference.

### FIELD OF THE INVENTION

The present invention relates to patient data-triggered insurance systems for providing risk sharing of risk events associated with elderly persons by providing a dynamic self-sufficient risk protection for the risk exposure components by means of an automated resource pooling system. In particular, the invention relates to automated event-driven systems triggering critical events on the patient dataflow pathway shortening the ability of an elderly person to live independently.

### BACKGROUND OF THE INVENTION

In today's lives, there is significant risk exposure related to many aspects in life and non-life sectors often resulting in an unexpected and dramatic change to the affected individual's life. In this context, risk-exposed units such as any kinds of objects, individuals, corporate bodies and/or legal entities, are necessarily confronted with many forms of active and passive risk management to hedge and protect against the risk of certain losses and events. In the prior art, one way to address such risk of loss is based on transferring and pooling the risk of loss from a plurality of risk-exposed entities to a dedicated pooling entity or system, In essence, this can be done by effectively allocating the risk of loss to this pooling unit or entity by pooling the resources of associated units that are exposed to a certain risk. In the event one of the units is hit by an event related to a transferred risk, the pooling entity directly intercepts the loss or damage caused by the event by transferring resources from the pooled resources to the affected unit. Resource pooling can be achieved by exchanging predefined quantities of resources with the resource pooling system; e.g., payments or premiums to be paid for the transfer of the risk. This means that predefined quantities of resources are exchanged for the other unit assuming the risk of loss. In the last decade, appropriate parameterization, dedicated capturing of related measuring parameters and adapted or self-adapting trigger-structures were the main focus for the industry in order to allow for providing a self-sufficient and automated operation of independent risk transfer systems.

For living individuals, a special kind of risk is based on the risk of loss of life and related possible losses; i.e., losses that occur as a consequence of the death of that individual. Such risks are traditionally handled by so-called life insurance systems, To administer a loss for benefits provided by a life insurance policy, a substantial amount of information must be collected and managed by the pooling entity in order to allow risk transfer. Appropriate documentation must be identified, captured and analyzed, such as death certificates or medical provider verification of a condition or service in the case of health/supplementary health. One important problem arises due to the fact that life insurance methods are triggered by the death of the unit, the risk of which is transferred. However, oftentimes problems arise for an individual before then, in that financial resources were threatened by losses occurring prior to death as a consequence of the events leading to the inability to lead an independent life. For elderly people, this is often the case when an individual suffers a serious elderly illness, such as heart attack, stroke, acute broken bone trauma, Alzheimer's disease, Parkinson's disease or any form of dementia, etc., resulting in the necessity of assisted living for the elderly person in order to maintain independent living. Typically, the patient is faced with increasing costs for medical treatment or other related costs, such as travel expenses or additional heating costs, as well as the decreasing ability to earn the money needed to meet their monthly financial needs. This may lead to the need to make many sacrifices; e.g., giving up independent living, not being able to provide sufficient financial support for care and/or selling their house. For especially elderly people, all of these financial concerns negatively affect their health. Recovery, if possible, is delayed and stress additionally aggravates the already poor health of the elderly person.

Long-term care models or assisted living have made up the fastest-growing segment of senior housing over the past several years. Despite this, there is substantial variation across countries and individuals about what “assisted living” is. However, there is a generally accepted definition on the key aspects of what constitutes assisted living. Typically, the key philosophical elements of assisted living include the following for assisted living in the person's private home as well as for assisted living in nursing homes or elderly people's residence: (a) services and oversight available 24 hours a day; (b) services to meet scheduled and unscheduled needs and facilitate aging in place; (c) care and services provided or arranged so as to promote independence; (d) an emphasis on consumer dignity, autonomy, and choice; and (e) an emphasis on privacy and a homelike environment, Yet there is considerable heterogeneity in the range of services offered and the populations served across facilities and markets. Indeed, many facilities fall short of the ideal, The demand for assisted living has evolved considerably over the past decade as facilities have come to serve a more disabled resident population with an increasingly complex array of services, potentially implying that assisted living could be a more viable nursing home alternative than it initially was. Growth in assisted living has been driven in large part by consumer preference. People who need assistance in performing everyday activities such as bathing, eating, or dressing prefer to receive supportive services in the least institutional and most homelike setting possible. A general population survey found that people would prefer to be cared for in an assisted living environment, i.e. the individual's private home, over a nursing home if they needed twenty-hour care, by a margin of six to one. In addition, for some people with less intensive care needs, it may be possible to purchase assisted living care at lower prices relative to nursing home care. Although the cost of assisted living can vary considerably depending on the amenities and services provided, various industry surveys put the average annual cost of assisted living care at around $30,000 to $40,000 in developed countries in 2009, compared to $70,000 to $80,000 per year for a semiprivate room in a nursing home. Up to now, efforts to collect assisted living supply data have largely been at the country level. Such data analysis has identified significant variation in assisted living supply across countries; however, examining potential within-state variation or correlations between assisted living supply and demand-level characteristics has not been possible, such that it was difficult to develop appropriate risk transfer systems for assisted living.

Conversely, as mentioned above, life insurance systems have been a valued technology for many years. Individuals, relatives and corporations have transferred their life risks to life insurance systems to protect themselves, their families and, in the case of officers and directors, their businesses from, inter alia, sudden loss of income. However, as a wage earner becomes older, the need to protect the family from sudden loss of the wage earner decreases or is eliminated. Alliteratively, in the case of a corporation, the officer or director on whose life a life insurance policy was issued may have retired or otherwise left the corporation, and the corporation no longer has a need for the policy. Up to now, the options for an elderly insured person were to allow the policy to lapse or, in the case of life insurance policies that were not paid up over time, continue to transfer the premiums, which in some cases are rather large, if coverage was still desired for some reason. However, new needs typically arise for the insured and possibly his/her family, as the insured grows older. For example, medical needs of terminally or chronically ill individuals may require large resources of cash or other liquid assets to pay for services that are not covered by the individual's health insurance or social security programs. In some cases, such individuals are best served by entering a nursing home or other assisted living facility where they can receive necessary, professional care on a regular basis. One of the problems is that when a terminally or chronically ill person, age 65 or older, wishes to enter a nursing home or other assisted living facility and further wishes to use social security programs to fund the person's stay and care, in many countries, state social security regulations generally require the person to divest himself or herself of substantially all liquid and liquidatable assets, subject to state-specific exemptions. Such regulations typically permit the person to retain a small amount of liquid or liquidatable assets. For example, countries generally limit the face value of life insurance of an assisted living, social security recipient to a small amount. Thus, in many cases, a considerable amount of a person's life insurance is vulnerable to divestment in order to receive social security funding allowing for covering assisted living expenses.

Assisted living expenses typically need for a large financial background.

Several methods exist to enable an elderly person to cover assisted living expenses, if he/she is not able to cover the expenses by his/her own liquid resources, which is rarely the case. First, if there is a life insurance risk transfer associated with the elderly person, the elderly person can simply cash in his or her policy for whatever cash value is in the policy. However, the cash value is often very small when compared to the costs of funding assisted living services and does not generally afford the social security applicant sufficient funds to pay for living expenses associated with residing in a nursing home, an assisted living center, a long-term care facility, or any other assisted living environment, as e.g. assisted living centers and especially assisted living services at the individual's private home. Moreover, due to the substantial nature of the costs associated with providing assisted living services, the cash value of the elderly person's life insurance policy is typically incapable of providing any significant delay in connection with the need for social security or other governmental assistance funds. In this case, the elderly person not only has to divest from his/her own assets, but also divest himself or herself of his/her life insurance.

To illustrate an example solution, in the United States, many insurance systems provide or afford the owner of a life insurance risk-transfer the opportunity to transfer any cash value or accelerated death benefit the owner has in the life insurance policy into a limited long-term care policy when the owner enters a nursing home or other assisted living facility. Although such accelerated death benefit systems have been slowly evolving in recent years, such systems typically can provide such risk transfer only for policies in which the owner is the insured and only when either the life expectancy of the insured is short, e.g., twelve months or less, or the insured' s illness, disease, or condition falls within certain specified categories. As an option, the elderly person holding the life insurance policy may submit the policy to a viatical or life settlement provider in an effort to obtain cash for the policy. Viatical settlements are liquation vehicles for life insurance policies in which a viatical settlement provider determines a life expectancy of the insured based on a variety of factors, including the medical history of the insured, and, based on the life expectancy and the face value of the policy, offers the owner of the policy a percentage of the face value of the policy, less any outstanding loans or presently due premiums. The proceeds to fund this often are acquired from investors (e.g., institutional or individual investors). Presently, the amount of a viatical settlement is often largely unregulated, although the cash payment made to the policy owner is required to be more than the cash value or accelerated death benefit, if any, of the policy. Some countries specify the percentage that must be paid to the policy owner if the insured has a life expectancy of twenty-four (24) months or less, Such specified percentages are typically eighty percent (80%) if the insured's life expectancy is less than six months, seventy percent (70%) if the insured's life expectancy is at least six months, but less than twelve months, sixty-five percent (65%) if the insured's life expectancy is at least twelve months, but less than eighteen months, and sixty percent (60%) if the insured's life expectancy is at least eighteen months, but less than twenty-four months, In exchange for the viatical settlement, the policy owner assigns or otherwise transfers his or her ownership of the life insurance policy to the viatical settlement provider, which in turn transfers the policy to the particular investor. It is worth to note that in different markets under different legal environments around the world, solutions can look very different to this US-specific solution.

Although in said methods some or all of the face value of a life insurance policy is available to the risk-transferring person, i.e. typically the policy owner, none of the methods require or ensure that the proceeds received by the policy owner are used to pay the living expenses of the policy owner while the policy owner resides at an assisted living facility. Since the policy owner or his/her designee has no obligation to use the liquidated or divested proceeds to cover assisted living expenses, state assistance programs, such as social security programs, often do not reap any benefit from the elderly person's divestiture of life insurance policies. In addition, there is currently no procedure for advising an individual elderly person as to his/her various options for divesting of his/her assets and life insurance policies to increase the assets used by that individual to pay assisted living expenses, and thereby temporarily defer his/her reliance on government assistance.

Apart from life insurance systems, risk transfer and resource pooling systems have been developed that cover so called critical illness systems, where the resource pooling unit operated by the insurer normally provides a lump sum cash payment if the risk-exposed unit, which is, from the insurer's perspective, the policyholder, is diagnosed with one of predefined critical illnesses as mutually negotiated and adjusted for the risk transfer between the risk transfer system and the risk-exposed unit, i.e., the individual. In the prior art, critical illnesses, which can typically be captured and covered by critical illness risk transfer systems, for example comprise heart attack, cancer, stroke and coronary artery by-pass surgery. Examples of other conditions that might be covered include: Alzheimer's disease, blindness, deafness, kidney failure, major organ transplant, multiple sclerosis, HIV/AIDS contracted by blood transfusion or during an operation, Parkinson's disease, paralysis of limb, terminal illness. One of the problems of these risk transfer system as provided by the prior art lies in the fact that the incidence of a condition may vary (i.e., increase or decrease) over time, and the diagnosis and treatment may improve over time, such that the financial need to cover some illnesses deemed critical a decade ago is no longer considered necessary today. Likewise, some of the conditions covered today may no longer need to be covered a decade or so from now. It is very difficult to adapt the prior art systems to such changing conditions without human interaction, What is clear is the fact that the financial hardship at the time of diagnosis and afterwards increases during the course of treatment, which seldom can be met by the prior art systems, Furthermore, operating the systems of the prior art requires a high level of human resources, because these systems cannot be adequately automated. Therefore, a large quantity of the pooled resources is used by the resource pooling system itself to administer the risk transfer, which makes the risk transfer expensive for the risk-exposed unit, Finally, another problem of traditional critical illness risk transfer systems comes from the fact that, due to medical progress, many patients covered by the critical illness risk transfer system no longer die, but can survive for many years after undergoing treatment for a heart attack, stroke and cancer. Due to the long survival period, such individuals, who were already struck once by a critical illness, continue to be exposed to the risk of a second or subsequent occurrence of a serious illness, In fact, especially with elderly persons. the risk typically does not decrease, since the health of these patients is already weakened by the first incidence of a serious illness. Since critical illnesses are traditionally meant to lead to death, the risk involving such individuals, who may be affected by a second or even more critical illnesses, which may lead to an even higher degree of inability to live independently, is no longer covered by the normal one-time payment of a critical illness risk transfer system. However, a combination of critical illnesses is precisely a characteristic feature of serious elderly illnesses leading to different levels of inability to live independently and not to direct death of the elderly person, Therefore, based on the typical one-time parametric payment transfer of critical illness system, although the patient survived his first bout with a serious illness, the elderly person may, at least financially, not survive the second time in dealing with the badly needed resources to keep up with the expenses for assistance for independent living, since this is typically associated with cost-intensive long-term care and services. This counts even more, since for the need of long-term care, one need not be “sick” in the traditional sense of the word or be “critically ill”. Though some of the latest critical illness risk-transfer systems may even comprise dedicated multiple payment features, the diagnosis of a critical illness is not the only reason for needing assistance of independent living. Often, assistance for independent living is need just due to general ageing and/or weakening of the ability to look after oneself (frailty). These cases completely fail to trigger the critical illness risk-transfer systems.

For risk transfer in relation to serious elderly illnesses, another major limiting feature of traditional critical illness insurance systems is related to mandatory boundary conditions for these systems given by age restrictions for critical illness risk transfer. Traditionally,. critical illness systems are only able to provide risk transfer for individuals by means of predefined schemes, e.g.,. a predefined child scheme restricted to the age of 30 days to 17 years, and for example a predefined adult scheme associated with risk transfer for individuals between 18 and 50 years, Above the upper limit (here 50 years), the system does not allow a new individual to pool resources in exchange for risk transfer of critical illness, In some systems, the actual risk coverage is longer, e.g., up to the age of 65 years, However, in those cases the individual must also have applied to the system before the first upper limit (here 50 years). For example. for the UK, individuals can usually apply for coverage up to the age of 65 with coverage expiring at 75. Therefore, the known system for critical illness risk transfer, equal to the typically predefined scheme of traditional life insurance risk transfer systems, are necessarily restricted to age conditions such as boundary requirements. Older individuals cannot be captured and covered by these systems anymore. Another disadvantage of the state of the art systems, which is especially relevant for risk transfer for serious elderly illness risk for older individuals, is already mentioned above. Advances in health care, especially in intensive care unit (ICU) healthcare, have enabled more especially elderly patients to survive common critical illnesses, and thus created a new population of elderly persons with serious elderly illness, who are chronically iii and incapable or disabled with respect to independent living without permanent or semi-permanent assistance. Patients with chronic elderly illness may for instance have persistent respiratory failure, dysfunction of other organs, and complications including neuropathy/myopathy, anemia, pressure ulcers, and recurrent infections. For example, it is conceivable that one class of chronic elderly illness can be identified by the placement of a tracheotomy for prolonged mechanical ventilation. It is a severe condition, imposing heavy burdens on patients, families, professional caregivers, and the healthcare system, Distressing symptoms are common, resource utilization and costs are enormous, return to the community is rare, and 6-month mortality rates exceed those for most malignancies. Another class of problematic chronic elderly illness is related to dementia.

Dementia is defined as a serious loss of global cognitive ability in a previously unimpaired person, beyond what might be expected from normal aging. it may be static, the result of a single global brain injury, or progressive, resulting in long-term decline due to damage or disease in the body. It bears mentioning that although dementia is far more common in the geriatric population (about 5% of those over 6.5 are said to be affected), it can occur before the age of 65, in which case it is called “early onset dementia”. Dementia is not a classical disease, but is typically indicated by set of non-specific symptoms, Affected cognitive areas can be memory, attention, language, and problem solving, Normally, symptoms must be present for at least six months to support a diagnosis. Cognitive dysfunction of shorter duration is called delirium. In advanced stages of dementia, subjects can be disoriented in time (not knowing the day, month, or even year), in place (not knowing where they are), and in person (not knowing who they and/or who others around them are). Dementia is classified as either reversible or irreversible, depending upon the etiology of the disease. it is important to note that dementia is not reversible in the sense that the system (human) undergoing the process can be returned to its original state, can be cured to a state without dementia. In the present state of neurological research, dementia is not curable as such. However, there can be specific conditions where the clinical symptoms mimic or closely mimic those suffered by those with dementia. The word reversible used in connection with dementia means that these conditions are reversible, However, as mentioned, that is not the case with dementia itself, and the condition/symptoms most likely will deteriorate over time, Despite the fact that there is no known cure, some treatments can potentially also slow down the process, Concerning reversibility, fewer than 10% of cases of dementia are due to causes that may be reversed with treatment, Some of the most common forms of dementia are: Alzheimer's disease, vascular dementia, frontotemporal dementia, semantic dementia and dementia with Lewy bodies, A patient can exhibit two or more dementing processes at the same time, as none of the known types of dementia protect against the others. About 10% of people with dementia have what is known as mixed dementia, which may be a combination of Alzheimer's disease and multi-infarct dementia, As for other serious elderly illnesses, like malignant cancer, also in the case of dementia, especially in advanced stages of dementia, the patient is exposed to the same problems as mentioned above for serious elderly illnesses. The patient will be faced with increasing costs for medical treatment or other related costs, and further with the decreasing ability to meet his/her financial needs. Cost estimations for treatment of the seriously ill elderly in the United States already exceed $20 billion and are increasing. Therefore, there is a need, especially for elderly people, to provide the possibility for a broadly applyable and automated risk transfer system related to acute and chronic serious elderly illnesses without an age restriction excluding the age group that needs risk transfer the most for illnesses and their chances to prolong independent and humane living.

There is a further problem explaining why traditional risk transfer systems related to life insurance risk transfer or critical illness risk transfer fail to provide appropriate mechanisms in relation to dementia as a serious elderly illness, i.e., resource pooling systems for risk transfer associated with the elderly. As mentioned, the traditional critical illness systems are triggered by the occurrence of the death of the person or the occurrence of a predefined critical illness. After the payout of an associated lump sum, the insured is not any more covered by the risk-transfer system. However, risk factors for dementia strongly increase after serious elderly illness diagnosis in elderly patients. Unfortunately, hospitalization increases the risk of a subsequent diagnosis of dementia. Studies show that illnesses requiring hospitalization and treatment in the intensive care unit (ICU) due to infection or severe sepsis, neurological dysfunction, such as delirium, or acute dialysis are all independently associated with an increased risk of a subsequent diagnosis of dementia (among others cf. C. Guerra et al., Risk factors for dementia after critical illness in elderly Medicare beneficiaries, Critical Care 2012, 16:R233). The studies show that over the three years of follow-up after the occurrence of a serious elderly illness, dementia was newly diagnosed in almost 18% of the patients who received intensive care and survived to hospital discharge. The results of these studies are significant, since even patients with previous indications of cognitive impairment for whom dementia could have been an escalation of a pre-existing condition were excluded from the studies (cf. FIG. 8, C. Guerra et al., Critical Care 2012 16:R233, doi:10.1186/cc11901). The studies clearly indicate that statistically, increasing age is very strongly associated with a diagnosis of dementia following ICU. The risk at 75 was more than double that of the 66 to 69 year olds. And this rose to mare than five times the risk for those ages 85 and older. Women had a marginally higher risk than men and, as other studies have shown, race was also an important risk factor. Length of stay in ICU was not a factor, nor was the need for mechanical ventilation (of. FIG. 5/6, C. Guerra et al., Critical Care 2012 16:R233). Three factors related to the serious elderly illnesses were able to be identified as being independently associated with an increased risk of a diagnosis of dementia (cf. FIG. 7, C. Guerra et al., Critical Care 2012 16:R233): a serious elderly illness with the presence of an infection, which increased to a higher risk with more severe infection such as severe sepsis; having acute neurologic dysfunction during the serious elderly illness, including anoxic brain damage, encephalopathy, and transient mental disorders; and finally, acute renal failure requiring dialysis. This last risk was time-dependent and only increased the risk 6 months after the patient had been discharged from the hospital. Thus, the possibility of returning to their homes sooner due to the availability of assisted living options typically improves the healing process of elderly people with serious elderly illnesses.

Therefore, with good reasons, older people often worry about dementia. While some risks are well known, for example alcoholism or stroke, the effects of illness are also significant. There is therefore a great need, in the state of the art, to provide automated risk transfer and resource pooling systems, especially for the elderly, also covering risks for acute and chronic serious elderly illnesses. These systems should be designable to be based on a single occurrence scheme following the diagnosis of a specific condition having multiple triggers following each diagnosis and each level of inability to live independently. As an option, the system should also be able to capture multiple occurrences of serious elderly illness, including dementia followed by the occurrence of another serious elderly illness, such as stroke or cardiac infarction, Traditional risk transfer systems, e.g., based on life insurance schemes or critical illness schemes, are not able to capture this group of elderly persons affected by serious elderly, chronic or acute illnesses and provide an efficient method for risk transfer, thereby providing mechanisms to unburden public social wellness services and duties and alleviate social hardship.

It bears mentioning that the above-mentioned correlation concerning dementia is exemplary for the elderly risk group. Other correlated serious illnesses for the elderly are well known. For example, compare the correlation of the risk for a stroke after coronary artery bypass (cf. S. Stamou et al,, Stroke After Coronary Artery Bypass, American Heart Association, Jan. 18, 2001). In summary, a strong need exists for a method of increasing resources available to an elderly person to at least partially fund living expenses of the elderly person at an assisted living facility or for assisted living at home, thereby delaying, even just for a short period, the individual's dependence upon government assistance or dependence on/need for care in an expensive private nursing facility, thereby prolonging his/her chance for independent living.

### SUMMARY OF THE INVENTION

It is an aim of the invention to provide a system and method for risk sharing of acute and/or chronic serious elderly illness risks associated with elderly persons by providing a dynamic self-sufficient risk protection for the risk exposure components via the elderly illness insurance system. The elderly illness insurance system, realized as an automated resource pooling system, shall be completely automated and self-adaptable/self-maintaining through its technical means and shall provide the technical risk transfer basis, which can be used by operators and/or service providers in the risk transfer or insurance technology for risk transfer related to serious elderly illness risks. A further aim of the invention provides for a way to technically capture, handle and automate complex related operations of the insurance industry related to elderly illness risk transfer. Another aim is to synchronize and adjust such operations based on technical means. In contrast to the standard approach, the resource pooling system shall create a reproducible operation with the desired, technically based, repetitious accuracy based on technical means, process flow and process control/operation, It is also an aim of the invention to provide a risk and resource pooling system able to cope with difficult chronic progress of elderly illnesses and further with complex related multiple risk events associated with a cohort of elderly persons.

According to the present invention, these aims are achieved particularly through the features of the independent claims, In addition, further advantageous embodiments follow from the dependent claims and the related description.

According to the present invention, the above-mentioned aims for risk transfer of serious illness risks associated with elderly persons are achieved, particularly, by providing a patient data driven system for multi-pillar triggered risk transfer associated with prolonged independent living under elderly illness occurrence by providing a dynamic self-sufficient risk protection for a variable number of risk exposure components by means of the resource pooling system, wherein the risk exposure components are connected to the resource pooling system by means of a plurality of payment-receiving modules configured to receive and store payments from the risk exposure components in order to pool their risks and resources, wherein the resource pooling system comprises a filter module for capturing age-related parameters of risk exposure components and for filtering risk exposure components associated with an age-related parameter greater than a predefined age threshold value by means of the predefined age threshold value, and wherein the resource pooling system provides automated risk protection for each of the connected risk exposure components based on received and stored payments and the age-related parameters of the risk exposure components triggering the predefined age threshold value, in that the resource pooling system generates a multi-dimensional trigger-table comprising a multi-layered data structure with predefined searchable acute and/or chronic elderly illnesses parameters for triggering by means of elderly illness triggers in the patient dataflow pathway, the predefined parameters of a first-layer data structure providing measuring parameters for the occurrence of serious elderly illnesses, and the predefined parameters of a second-layer data structure providing measuring parameters for the occurrence of an acute broken bone trauma, and the predefined parameters of a third-layer data structure providing measuring parameters for long-term care indications, and the predefined parameters of a fourth-layer data structure providing measuring parameters for assisted living indications, and the predefined parameters of a fifth-layer data structure providing measuring parameters for extended assistance indications, in that the resource pooling system comprises an event-driven core engine comprising elderly illness triggers triggering the measurement of values in patient dataflow pathways of the connected risk exposure components, wherein, if an occurrence of an acute or chronic elderly illness on the patient dataflow pathway of a risk exposure component is triggered by exceeding one of the predefined searchable acute and/or chronic elderly illnesses parameters, a corresponding trigger-flag is set by means of the resource pooling system and a parametric transfer of payments is assigned to this corresponding trigger-flag, wherein a loss associated with the acute and/or chronic elderly illness is distinctly covered by the resource pooling system based on the respective trigger-flag arid based on the received and stored payment parameters from risk exposure components by at least one parametric payment transfer from the resource pooling system to the risk exposure component. The elderly illness triggers can for example comprise a trigger for triggering the occurrence of measuring parameters indicating dementia based on measuring parameters associated with the permanent clinical loss of the ability to remember and/or reason and/or perceive, understand, express arid give effect to ideas in the patient dataflow pathway. The triggered measuring parameters indicating dementia can comprise physical parameters and/or psychological parameters and/or biochemical parameters and/or cognitive factors based on adrenal exhaustion factors and/or food and chemical reactions factors and/or nutritional deficiency factors and/or stress factors and/or depression factors, and/ or denial factors, indicating confirmed impairment of cognitive functions. The predefined parameters of the first-layer data structure providing measuring parameters for the occurrence of serious elderly illnesses can further comprise at least parameters indicating the occurrence of dementia, heart attack, cancer, stroke, coronary artery by-pass surgery, Alzheimer's disease, blindness, deafness, kidney failure, major organ transplant, multiple sclerosis, HIV/AIDS contracted by blood transfusion or during an operation, Parkinson's disease, paralysis of limb or terminal illness in the patient dataflow pathway. The predefined parameters of the first-layer data structure providing measuring parameters for the occurrence of serious elderly illnesses can comprise at least elderly illness trigger parameters for triggering the occurrence of measuring parameters indicating dementia based on measuring parameters associated with the permanent clinical loss of the ability to remember and/or reason and/or perceive, understand, express and give effect to ideas in the patient dataflow pathway. Thus, the measuring and triggering of parameters relating to mobility/physical abilities are extended to parameters measuring permanent clinical losses. The patient dataflow pathway may also for example be monitored by the resource pooling system by capturing patient-measuring parameters of the patient dataflow pathway at least periodically and/or within predefined time frames or periods. Finally, the patient dataflow pathway can for example be dynamically monitored by the resource pooling system in that it triggers patient--measuring parameters of the patient dataflow pathway transmitted from associated measuring systems, The invention has, inter alia, the advantage that the system provides the technical means to meet elderly persons needs related to the financial hardships at the time of the diagnosis of a chronic and/or acute serious elderly illness, which will typically become more acute as the treatment progresses. Therefore, dementia, stroke, heart attack or cancer fears, etc., and the related consequences that are suffered by many elderly persons can be addressed with the automated resource pooling system according to the invention. The system furthermore has the advantage that smaller payments than in traditional risk-transfer systems are sufficient to allow for a safe operation of the system. The operational aspects of the system are transparent for operators as well as covered risk units, since payment is transferred in response to certain triggers on the elderly patient's information pathways. The system is able to provide an adaptable survival period for elderly persons to be confirmed or defined by the risk transfer. The system is further able to provide the technical implementation of an automated system that is based on a drawdown payment operation or a predefined payment operation. The system also provides the technical means, which can support various assisted living benefits and assistance services, initial long-term care (LTC), acute broken bone trauma and serious elderly illnesses such as dementia, and/or stroke, and/ or heart attack, and/or cancer. Further, in contrast to the present invention, many prior art systems fail to take over risk transfers if the individual suffered from the insured condition before the risk transfer was initiated (this is known as the Pre-Existing Condition Exclusion), or because the individual suffered from a condition that led to a claim under the risk transfer of the insured illnesses—for example, it was known that an individual suffered from high blood pressure before the risk transfer was activated, and suffered a stroke after the risk transfer had been activated. Pre-Existing Condition Exclusion typically covers first heart attack and/or stroke, wherein for simplified underwriting, in order to initiate risk-transfer, previous heart attacks or strokes will be captured under this boundary condition. Another advantage of the system is based on the fact that payments are directly transferred to the risk transfer unit or the elderly person. In a preferred embodiment variant, the inventive system (respectively, the multi-layered data structure with the predefined searchable trigger parameters triggering the trigger parameters in the patient dataflow pathway (213, 223, 233) by means of elderly illness triggers (31, . . . , 35)) comprise at least the predefined parameters of the fourth-layer data structure (74) providing measuring parameters for assisted living indications, and the predefined parameters of the fifth-layer data structure (75) providing measuring parameters for extended assistance indications. I.e. in this embodiment variant, these two components form the integral part to the system, which is in case of the fourth-layer data structure (74) triggering for assisted living benefit (e.g. providing care benefits and home adaptions in one's private home) and in case of the fifth-layer data structure (75) triggering for assistance services (e.g. concierge services to arrange independent care advice), while the other three layered data structures (71, 72, 73) are optionally realizable for this embodiment variant.

In one alternative embodiment, the receiving and preconditioned storage of payments from risk exposure components for the pooling of their risks is dynamically determined based on total risk and/or the likelihood of the risk exposure of the pooled risk exposure components. This alternative embodiment has, infer the advantage that the operation of the resource pooling system can be dynamically adapted to changing conditions of the pooled risk, for example, changing demographic conditions or changing age distributions or the like of the pooled risk components, i.e., elderly persons. A further advantage is that the system needs no manual adaption when it is operated in different environments, places or countries, because the size of the payments of the risk exposure components is directly related to the total pooled risk.

In another alternative embodiment, the number of pooled risk exposure components is dynamically adapted, by means of the resource pooling system, to a range where non-covariant occurring risks covered by the resource pooling system affect only a relatively small proportion of the total pooled risk exposure components at a given time. This alternative has, inter alia, the advantage that the operational and financial stability of the system can be improved.

In a further alternative embodiment, the elderly illness triggers are dynamically adapted by means of an operating module based on time-correlated incidence data for a serious elderly illness condition and/or diagnosis or treatment conditions indicating improvements in diagnosis or treatment. This alternative has, inter alia, the advantage that improvements in diagnosis or treatment can be dynamically captured by the system and dynamically affect the overall operation of the system based on the total risk of the pooled risk exposure components.

In yet another alternative embodiment, a plurality of parametric payments are leveled by a predefined total payment sum determined at least based on the risk-related component data and/or on the likelihood of the risk exposure for one or a plurality of the pooled risk exposure components based on the risk-related component data, and wherein a first portion is transferred up to predefined percentage of said total payment sum and the following portions are transferred up to the residual part given by said total payment sum. The predefined total payments con tor example be leveled to any appropriate lump sum, such as $50,000 up to $500,000, for instance, or any other sum related to the total transferred risk and the amount of the periodic payments of the risk exposure component. As an alternative embodiment of the system, the elderly illness trigger can for example comprise multi-dimensional trigger channels, wherein each of said trigger-flags is assigned to a first dimension trigger channel, comprising a first trigger-level triggering occurrence parameters of the need for assisted living benefit, a second trigger-level triggering serious elderly illness parameters, a third trigger-level triggering acute broken bone trauma parameters, a fourth trigger-level triggering an initial long-term care treatment phase, and a fifth trigger-level triggering needs for assistance services, and each of said trigger-flags is assigned to at least a second or higher dimension trigger channel, and comprises additional trigger-stages based on the first to fifth trigger-level of the multi-dimension or multi-pillar trigger channel. However, please note, that a man skilled in the art understands that the ordering, as exemplary given herein, is just an example, whereas restructuring the order of the trigger-levels and/or trigger channels does not change the core of the inventive system. Thus, the serious elderly illness trigger can also comprise multi-dimensional trigger channels, wherein each of said trigger-flags is assigned to one of the five dimensions. This alternative, inter alia, has the advantage that the draw-down payments or the payments of predefined amounts, which depend on one of the five trigger pillars, i.e., the different stages of triggers, allow for an adapted payment of the total sum that is dependent on the stage of the serious elderly illness as triggered by the system, i.e., the inability to live independently and need for assistant living support.

In one alternative embodiment, a periodic payment transfer from the risk exposure components to the resource pooling system via a plurality of payment receiving modules is requested by means of a monitoring module of the resource pooling system, wherein the risk transfer or protection for the risk exposure components is interrupted by the monitoring module when the periodic transfer is no longer detectable by means of the monitoring module, As an alternative, the request for periodic payment transfer can be interrupted automatically or waived by means of the monitoring module, when the occurrence of indicators for elderly illness is triggered in the patient dataflow pathway of a risk exposure component. These alternative embodiments have, inter alia, the advantage that the system allows for a further automation of the monitoring operation, especially of its operation with regard to the pooled resources.

In a further alternative embodiment, an independent verification elderly illness trigger of the resource pooling system is activated if indicators are triggered for elderly illness in the patient dataflow pathway of a risk exposure component by means of the elderly illness trigger and wherein the independent verification elderly illness trigger additionally is triggering the occurrence of indicators regarding elderly illness in an alternative patient dataflow pathway with independent measuring parameters from the primary patient dataflow pathway in order to verify the occurrence of the elderly illness at the risk exposure component. As an alternative, the parametric drawdown transfer of payments is only assigned to the corresponding trigger-flag if the occurrence of the elderly illness at the risk exposure component is verified by the independent verification elderly illness trigger. These alternative embodiments have, inter alia, the advantage that the operational and financial stability of the system can thus be improved. In addition, the system is rendered less vulnerable to fraud and counterfeit.

In addition to the system as described above and the corresponding method, the present invention also relates to a computer program product that includes computer program code means for controlling one or more processors of the control system such that the control system performs the proposed method; and it relates, in particular, to a computer program product that includes a computer-readable medium containing the computer program code means for the processors.

### BRIEF DESCRIPTION OF THE DRAWINGS

The present invention will be explained in more detail, by way of example, with reference to the drawings, in which:

FIG. 1 shows a block diagram schematically illustrating an exemplar/parametric, event-driven elderly illness insurance system based on a resource pooling system 1 according to the invention for risk sharing of elderly illness risks associated with elderly persons by providing dynamic self-sufficient risk protection for a variable number of risk exposure components 21, 22, 23, said elderly persons. The resource pooling system 1 comprises an assembly module 5 to process risk-related component data 211, 221, 231 and to provide the likelihood 212, 222, 232 of said risk exposure for one or a plurality of the pooled risk exposure components 21, 22, 23, wherein the risk exposure components 21, 22, 23 are connected to the resource pooling system 1 by means of a plurality of payment receiving modules 4 that are configured to receive and store 6 payments 214, 224, 234 from the risk exposure components 21, 22, 23 for the pooling of their risks, and wherein the resource pooling system 1 comprises an event-driven core engine 3 that comprises elderly illness triggers 31, . . . , 35, which trigger a patient dataflow pathway 213, 223, 233 to provide risk protection for a specific risk exposure component 21, 22, 23. The patient dataflow pathway 213, 223, 233 is monitored by the resource pooling system 1 in that patient measuring parameters of the patient dataflow pathway 213, 223, 233 are captured, wherein the patient dataflow pathway 213, 223, 233 is dynamically monitored and triggered for patient measuring parameters of the patient dataflow pathway 213, 223, 233, which is transmitted from associated measuring systems.

### DETAILED DESCRIPTION OF THE PREFERRED EMBODIMENTS

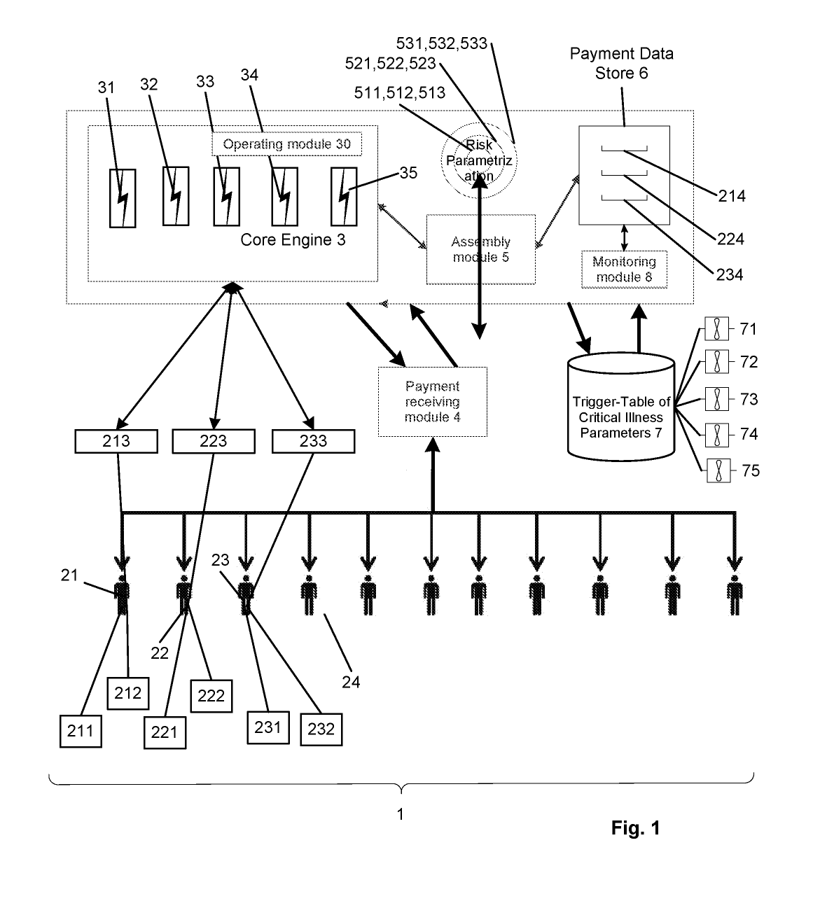

FIG. 1 schematically illustrates an architecture for a possible implementation of an embodiment of the parametric, event-driven resource pooling system 1 for risk sharing of elderly illness risks associated with elderly persons. In FIG. 1, reference numeral 1 refers to the resource pooling system for risk sharing of the risk exposure components 21, 22, 23, etc. The resource pooling system 1 provides a dynamic self-sufficient risk protection and corresponding risk protection structure for a variable number of risk exposure components 21, 22, 23, i.e., elderly persons or individuals, by its means. The system 1 includes at least one processor and associated memory modules. The system 1 can also include one or more display units and operating elements, such as a keyboard, and/or graphical pointing devices, such as a computer mouse. The resource pooling system 1 is a technical device comprising electronic means that can be used by service providers in the field of risk transfer or insurance technology for risk transfer related to elderly illness risks and related to prolonging the chance for independent living through assisted living or the like. The invention seeks to technically capture, handle and automate complex related operations of the insurance industry. Another aspect is to synchronize and adjust such operations based on technical means. In contrast to the standard approach, the resource pooling system also achieves a reproducible operation with the desired technical, repetitious accuracy because it is completely based on technical means, process flow and process control operation.

The resource pooling system 1 comprises an assembly module 5 to process risk-related component data 211, 221, 231 and to provide the likelihood 212, 222, 232 of said risk exposure for one or a plurality of the pooled risk exposure components 21, 22, 23, etc. based on the risk-related component data 211, 221, 231. The resource pooling system 1 can be implemented as a technical platform, which is developed and implemented to provide elderly illness risk transfer through a plurality of (but at least one) payment receiving module 4. The risk exposure components 21, 22, 23, etc. are connected to the resource pooling system 1 by means of the plurality of payment receiving modules 4 configured to receive and store payments 214, 224, 234 from the risk exposure components 21, 22, 23, etc. to pool their risks in a payment data store 6. As a variant, the transferred and pooled payments can be grouped to provide risk-transfer for a defined group of risk exposure components 21, 22, 23. The plurality of risk exposure components 21, 22, 23 from a cohort of selected elderly persons, where during capturing the risk exposure components 21, 22, 23 to be pooled by the system 1, age-related parameters of risk exposure components are captured. Based on the captured age-related parameters, the risk exposure components are filtered using a filter module, wherein by means of the filter module, only risk exposure components 21, 22, 23 associated with an age-related parameter greater than a predefined age-threshold value are allowed to be pooled by the system 1. The predefined age-threshold value can for example be set to 50 years or another appropriate age allowing the selection of a specific cohort of elderly persons. As an alternative embodiment, the selection criterion can comprise further parameters such as gender, origin, habits, urban or rural conglomeration, etc.

The storage of the payments can be implemented by transferring and storing component-specific payment parameters. The payment amount can be dynamically determined by means of the resource pooling system I based on total risk of the overall pooled risk exposure components 21, 22, 23. For the pooling of the resources, the resource pooling system 1 can comprise a monitoring module 8 requesting a periodic payment transfer from the risk exposure components 21, 22, 23, etc. to the resource pooling system 1 by means of the plurality of payment receiving modules 2, wherein the risk protection for the risk exposure components 21, 22, 23, etc. is interrupted by the monitoring module 8, when the periodic transfer is no longer detectable by means of the monitoring module 8. In one alternative embodiment, the request for periodic payment transfers is automatically interrupted or waived by means of the monitoring module 8, if the occurrence 1001 of indicators for one of the predefined elderly illnesses 71, . . . , 75 of the multi-dimensional trigger-table 7 is triggered in the patient dataflow pathway of a risk exposure component 21, 22, 23, etc. The resource pooling system 1 further comprises said predefined multi-dimensional table 7 comprising a multi-layered data structure with predefined searchable acute and/or chronic elderly illness 71, . . . , 75 parameters for triggering by means of elderly illness triggers 31, . . . , 35 in the patient dataflow pathway 213, 223, 233. It is to be noted that at least one of the elderly illness triggers 31, . . . , 35 is based upon the classification of a risk exposure components 21, 22, 23, i.e. an elderly person, as being within a particular “state”, i.e. parameter state, having lost the ability to do certain physical activities due to the effect of ageing. Thus, measuring parameters indicating one of said acute and/or chronic elderly illnesses 71, . . . , 75, do not need to indicate a medical illness” at the same time, but can be simply a consequence of the ageing of a risk exposure component 21, 22, 23. The predefined parameters of a first-layer data structure 71 of said predefined multi-dimensional table 7 provide measuring parameters for the occurrence of serious elderly illnesses. The predefined parameters of a second--layer data structure 72 of said predefined multi-dimensional table 7 provide measuring parameters for the occurrence of an acute broken bone trauma. The predefined parameters of a third-layer data structure 73 of said predefined multi-dimensional table 7 provide measuring parameters for long-term care indications. The predefined parameters of a fourth-layer data structure 74 of said predefined multi-dimensional table 7 provide measuring parameters for assisted living indications. The predefined parameters of a fifth-layer data structure 75 of said predefined multi-dimensional table 7 provide measuring parameters for extended assistance indications. The trigger parameters of acute and/or chronic elderly illnesses 71, . . . , 75 parameters can indicate the occurrence of dementia and/or heart attack and/or cancer and/or a stroke and/or coronary artery by-pass surgery, Alzheimer's disease and/or blindness and/or deafness and/or kidney failure and/or major organ transplant and/or multiple sclerosis and/or HIV/AIDS contracted by blood transfusion or during an operation and/or

Parkinson's disease and/or paralysis of limb and/or terminal illness and/or any other definable and measurable elderly illnesses in the elderly patient dataflow pathway 213, 223, 233. The latter can simply comprise the “need for care” as measured by the ability to perform basic activities of daily living (e.g. washing, dressing, eating), i.e. is not a classical illness, but a measurable defined risk-transfer event (insured event). The triggers 31, . . . , 35 are uni- or bidirectionally connected with the predefined multi-dimensional table 7 of elderly illnesses 71, . . . , 75, wherein the triggering 31, . . . , 35 is performed based on the elderly illness 71, . . . , 75 parameters stored in the predefined multi-dimensional table 7 by means of the multi-layered data structure. The elderly illness triggers 31, . . . , 35 can comprise a trigger for triggering the occurrence of measuring parameters indicating dementia based on measuring parameters associated with the permanent clinical loss of the ability to remember and/or reason and/or perceive, understand, express and give effect to ideas in the patient dataflow pathway 213, 223, 233. The elderly illness triggers 31, . . . , 33 can further comprise a trigger for triggering measuring parameters as exclusion parameters, indicating e.g. alcohol and/or drug abuse in the patient dataflow pathway 213. 223, 233, and/or triggering ABS (atomic, biological or chemical weapons) terrorism and/or active participation in war etc., wherein upon triggering measuring parameters indicating alcohol and/or drug abuse, the related risk exposure component (21, 22, 23, . . . ) is rejected from pooling of the risk and resources by means of the resource pooling system 1.

As also schematically illustrated in FIG. 1, the resource pooling system 1 includes a data storing module for capturing the risk-related component data and multiple functional modules; e.g., the payment receiving modules 4, the core engine 3 with the triggers 31, . . . , 33, the assembly module 5 or the operating module 30. The functional modules can be implemented at least partly as programmed software modules stored on a computer-readable medium, connected as fixed or removable means to the processor(s) of system 1 or to associated automated systems. One skilled in the art understands, however, that the functional modules can also be fully implemented by means of hardware components, units and/or appropriately implemented modules. As illustrated in FIG. 1, the system 1 can be connected via a network, such as a telecommunications network, to the payment-receiving module 4. The network can include a wired or wireless network; e.g., the Internet, a GSM network (Global System for Mobile Communication), a UMTS network (Universal Mobile Telecommunications System) and/or a WLAN (Wireless Local Region Network), and/or dedicated point-to-point communication lines. In any case, the technical electronic money schemes for the present system comprise adequate technical, organizational and procedural safeguard means in order to prevent. contain and detect threats to the security of the scheme, particularly the threat of counterfeits. The resource pooling system 1 further comprises all necessary technical means for electronic money transfer and association, for example initiated by one or more associated payment-receiving modules 4 over an electronic network. The monetary parameters can be based on all possible electronic and transferable means such as e-currency, e-money, electronic cash, electronic currency, digital money, digital cash, digital currency, or cyber currency, etc., which can only be exchanged electronically. The payment data store 6 provides the means for associating and storing monetary parameters associated with a single one of the pooled risk exposure components 21, 22, 23. The present invention can involve the use of the aforementioned networks, such as computer networks or telecommunications networks, and/or the internet and digital stored value systems. Electronic funds transfer (EFT), direct deposit, digital gold currency and virtual currency are further examples of electronic money. Also, the transfer can involve technologies, such as financial cryptography and technologies enabling the same. For the transaction of the monetary parameters, it is preferable for hard electronic currency to be used without the technical possibilities for disputing or reversing any charges. The resource pooling system 1 for example supports non-reversible transactions, The advantage of this arrangement is that the operating costs of the electronic currency system are greatly reduced by not having to resolve payment disputes. However, this way, it is also possible for electronic currency transactions to clear instantly, making the funds available to the system 1 immediately. This means that using hard electronic currency is more akin to a cash transaction, However,. the use of soft electronic currency is also conceivable, such as currency that allows for the reversal of payments, for example having a “clearing time” of 72 hours, or the like. The electronic monetary parameter exchange method applies to all connected systems and modules related to the resource pooling system 1 of the present invention, such as the payment receiving module 4. The monetary parameter transfer to the resource pooling system 1 can be initiated by a payment-receiving module 4 or on request by the resource pooling system 1.

The resource pooling system 1 comprises an event-driven core engine 3 comprising elderly illness triggers 31, . . . , 35 for triggering component-specific measuring parameters in the patient dataflow pathway 213, 223, 233 of the assigned risk exposure components 21, 22, 23, etc., i.e., the insured elderly person, where insured means a person transferring risk to a unit in exchange for resources, The patient dataflow pathway 213, 223, 233 can for example be monitored by the resource pooling system 1, capturing patient-related measuring parameters of the patient dataflow pathway 213, 223, 233 at least periodically and/or within predefined time periods. The patient dataflow pathway 213, 223, 233 can for example also be dynamically monitored by the resource pooling system 1, by triggering patient-measuring parameters of the patient dataflow pathway 213, 223, 233 transmitted from associated measuring systems. By triggering the patient dataflow pathway 213, 223, 233, which comprises dynamically recorded measuring parameters of the concerned risk exposure components 21, 22, 23, etc., the system 1 is able to detect the occurrence of an elderly illness and dynamically monitor the different stages during the progress of the elderly illness in order to provide appropriately adapted and gradated risk protection for a specific risk exposure component 21, 22, 23, etc. Such a risk protection structure is based on received and stored payments 214, 224, 234 from the related risk exposure component 21, 22, 23, etc. and/or related to the total risk of the resource pooling system, 1 based on the overall transferred elderly illness risks of all pooled risk exposure components 21, 22, 23, etc.

In FIG. 1, the block diagram shows the possible trigger stages, wherein the elderly illness triggers 31, . . . , 35 can for example comprise a trigger for triggering the occurrence of the measuring parameters, indicating a heart attack and/or cancer and/or a stroke and/or coronary artery by-pass surgery in the patient dataflow pathway 213, 223, 233. Further, the elderly illness triggers 31, . . . , 35 can comprise a trigger for triggering the occurrence of measuring parameters indicating Alzheimer's disease, dementia, blindness, deafness, kidney failure, major organ transplant, multiple sclerosis, HIV/AIDS contracted by blood transfusion or during an operation, Parkinson's disease, paralysis of limb, terminal illness in the patient dataflow pathway 213, 223, 233. A significant portion of cases of occurrences of elderly illness can be related to heart attack, stroke and cancer, and dementia, The average age of individuals 21, 22, 23 at which an elderly illness can be detected in the patient dataflow pathway 213, 223, 233 is for instance over 50 years, depending on the system's definition of the pooled elderly persons; however, this depends on the development of diagnostic and other medical means. Normally, the statistics obey the same factors for all countries where statistics are maintained. However, it is of great concern to observe the increasing number of elderly illness occurrences—particularly regarding prolonging independent living cases.

Earlier diagnosis due to better diagnostic equipment may be partially responsible for these figures. Therefore, to ensure proper operation of the resource pooling system 1, the definitions of the stored trigger parameters 71, . . . , 75 of elderly illness in the trigger table 7 can be dynamically adapted based on a monitoring of changing risks in the risk exposure components 21, 22, 23. In particular, the trigger parameters 71, . . . , 75 can be region-specific, country-specific and/or specific of the total pooled risk, adapted or changed. New elderly illnesses 71, . . . , 75 can be added, while others can be deleted from the triggerable list of elderly illnesses by the resource pooling system, owing to better treatments or other changed environmental conditions. In one alternative embodiment, the elderly illness triggers 31, . . . , 33 can be dynamically adapted by means of an operating module 30, based on time-correlated incidence dates of an elderly illness condition and/or diagnosis or treatment conditions indicating improvements in diagnosis or treatment.

In addition to the adaptation of the triggers 31, . . . , 35, the amount of the payments requested from the risk exposure components 21, 22, 23 can be adjusted accordingly by the resource pooling system 1. Therefore, the receiving and preconditioned storage 6 of payments 214, 224, 234 from risk exposure components 21, 22, 23, etc. for the pooling of their risks can be determined dynamically, based on total risk 50 and/or the likelihood of the risk exposure of the pooled risk exposure components 21, 22, 23, etc. To improve operational and functional security of the resource pooling system 1 even further, the number of pooled risk exposure components 21, 22, 23, etc. can be dynamically adapted by means of the resource pooling system 1 to a range where non-covariant, occurring risks covered by the resource pooling system 1 affect only a relatively small proportion of the total pooled risk exposure components 21, 22, 23, etc. at a given time.

The total risk 50 of the pooled risk exposure components 21, 22, 23, etc. can comprise several risk contributions, as it can comprise a first risk contribution 511 of each pooled risk exposure component 21, 22, 23, etc. that is associated with risk exposure in relation to the occurrence of serious elderly illnesses, a second contribution 521 of each pooled risk exposure component 21, 22, 23, etc. that is associated with the occurrence of an acute broken bone trauma, a third contribution 531 of each pooled risk exposure component 21, 22, 23, etc. that is associated with the need for long-term care, a fourth contribution 541 of each pooled risk exposure component 21, 22, 23, etc. that is associated with the need for assisted living support, and a fifth contribution 551 of each pooled risk exposure component 21, 22, 23, etc. that is associated with the need for extended assistance. The risk contributions 511, . . . , 551 are reflected by the multi-dimensional trigger-table 7 with the multi-layered data structure with predefined searchable acute and/or chronic elderly illnesses 71, . . . , 75 parameters. The triggering parameters 71, . . . , 73 of the covered elderly illnesses are comprised and stored in a predefined multi-dimensional table 7, such as an appropriately structured hash table of elderly illnesses 71, . . . , 75, respectively elderly illness parameters 71, . . . , 75. The elderly illness losses occur as a consequence of the triggering, respectively diagnosis of risk exposure components 21, 22, 23, etc. with regard to one of the searchable elderly illnesses; i.e., the possible need of a risk exposure component 21, 22, 23, etc. to be covered by the pooled resources of the resource pooling system 1 is linked to the risk of the occurrence of on elderly illness requiring complex medical treatment and handling, The total risk 50 of the pooled risk exposure components 21, 22, 23, etc. can also comprise further risk contribution not mentioned here; i.e., up to the i-th risk contribution, associated with risk exposure in relation to a second and/or subsequent elderly illness(es).

As mentioned, the resource pooling system 1 comprises an event-driven core engine 3 comprising elderly illness triggers 31, . . . , 35 triggering measuring values in patient dataflow pathways 213, 223, 233 of the connected risk exposure components 21, 22, 23, etc. If an occurrence of an acute or chronic elderly illness 71, . . . , 75 on the patient dataflow pathway 213, 223, 233 of a risk exposure component 21, 22, 23, . . . is triggered by exceeding one of the predefined searchable acute and/or chronic elderly illnesses 71, . . . , 75 parameters, a corresponding trigger-flag is set via the resource pooling system 1 and a parametric transfer of payments is assigned to that corresponding trigger-flag, wherein a loss associated with the acute or chronic elderly illness 71, . . . , 75 is covered separately by the resource pooling system 1 based on the respective trigger-flog and based on the received and stored payment parameters 214, 224, 234 from risk exposure components 21, 22, 23 by at least the one parametric payment transfer from the resource pooling system 1 to the risk exposure component 21, 22, 23, etc. The parametric payment transfer can comprise electronic and non-electronic cash transfer as well as non-cash payment transfers, as e.g. provisions of services or tangible goods. If multiple occurrences of a second or subsequent elderly illness 71, 72, 73 are triggered on the patient dataflow pathway 213, 223, 233 of a risk exposure component 21, 22, 23, i.e., if triggering of an occurrence of a first or second or subsequent elderly illness 71, . . . , 75 goes into effect in the patient dataflow pathway 213, 223, 233, thereby worsening the ability of the elderly person to live independently, respectively increasing his/her need for assisted living, adapted trigger-flags are set via the resource pooling system l and an adapted parametric draw-down or predefined transfer of payments is assigned to this corresponding trigger-flag. A loss associated with the first or second or subsequent elderly illness(es) 71, . . . , 73 is separately covered by the resource pooling system 1, based on the respective trigger-flag and the received and stored payment parameters 214, 224, 234 from risk exposure components 21, 22, 23 by the parametric draw-down or predefined transfer from the resource pooling system 1 to the risk exposure component 21, 22, 23, etc. The payment receiving module 4 can, as an input device, comprise one or more data processing units, displays and other operating elements, such as a keyboard and/or a computer mouse or another pointing device. As mentioned previously, the receiving operation of the payments with regard to the risk exposure components 21, 22, 23 is monitored based on the stored component-specific payment parameters in the payment data store 6. The different components of the resource pooling system 1, such as the payment receiving module 4 with the core engine 3 and the assembly module 5, can be connected via a network for signal transmission. The network can for example comprise a telecommunications network, such as a wired or wireless network, e.g., the Internet, a GSM network (Global System for Mobile Communications), a UMTS network (Universal Mobile Telecommunications System) and/or a WLAN (Wireless Local Area Network), a Public Switched Telephone Network (PSTN) and/or dedicated point-to-point communication lines. The payment receiving module 4 and/or core engine 3 and the assembly module 5 can also comprise a plurality of interfaces for connecting to the telecommunications network adhering to the transmission standard or protocol. As an alternative embodiment, the payment receiving module 4 can also be implemented as an external device relative to the resource pooling system 1, which provides the risk transfer service via the network for signal transmission, e.g., by a secured data transmission line.

A parametric payment is transferred by triggering the occurrence of the elderly illness 71, . . . , 75 by means of the elderly illness trigger of the core engine 3, thus triggering the measuring parameters of the specific risk exposure component 21, 22, 23 in the related patient dataflow pathway 213, 223, 233. The first, second and third parametric payments can be denoted in “units” operationally defined by means of the risk-transfer system 1. The amount of those units can be either set as fixed running parameters of the system 1 for the duration of the transferred risks or any other defined time frame, or dynamically adapted based upon possibly changing environmental boundary conditions, such as medical or therapeutic costs, or based upon the total pooled resources by means of the system 1. The changing of the environmental boundary conditions can be triggered dynamically or captured by the system 1. One “unit” can be assigned to correspond to an equivalent in a specific currency (e.g., Euros, dollars or Swiss francs). The core engine 3, analogously to the resource pooling system 1 and the other components of the system, is implemented based on underlying electronic components, steering codes and interacting interface devices, such as appropriate signal generation modules or other modules interacting electronically by means of appropriate signal generation between the different modules, devices, or the like. For example, a parametric payment of 10,000 can be transferred by triggering, by means of the first pillar elderly illness trigger 31, the occurrence of measuring parameters indicating the occurrence of a serious elderly illness, such as first heart attack, stroke, malignant cancer, smaller incidence of ductal carcinoma in situ (DCIS), early prostate carcinoma, dementia, etc. Another parametric payment can be transferred by triggering, by means of the second pillar elderly illness trigger 32, the occurrence of measuring parameters indicating the occurrence of an acute broken bone trauma, wherein the parametric payment comprises 1,000 per accident once per year, for hip or limb fracture with a maximum benefit of 5,000. A further parametric payment can be transferred by triggering, by means of the third pillar elderly illness trigger 33, the occurrence of measuring parameters indicating the need for initial long-term care, wherein the parametric payment for example comprises up to 2,500 per month to cover care costs when going into a care home for a maximum of six months. Another parametric payment can be transferred by triggering, by means of the fourth pillar elderly illness trigger 34, the occurrence of measuring parameters indicating the need for extended assistance services, concierge services to arrange independent care advice, etc. Finally, a further parametric payment can be transferred by triggering, via the fifth pillar elderly illness trigger 35, the occurrence of measuring parameters indicating the need for assisted living covers, wherein the parametric payment can comprise 18,000 for care at private home benefits and home adaption, with a maximum care at private home benefit payout of 1,000 per month up to a total assigned parametric amount and adaptions of up to 10,000. If a care at private home benefit is received, there may be a further maximum of 2,000 for respite care.

In the case of dementia, the elderly illness triggers 31, . . . , 35 can comprise a trigger for triggering the occurrence of measuring parameters indicating dementia based on measuring parameters associated with the permanent clinical loss of the ability to remember and/or reason and/or perceive, understand, express and give effect to ideas in the patient dataflow pathway 213, 223, 233. The triggering measuring parameters indicating dementia can also comprise physical parameters and/or psychological parameters and/or biochemical parameters and/or cognitive factors based on adrenal exhaustion factors and/or food and chemical reaction factors and/or nutritional deficiency factors and/or stress factors and/or depression factors, or denial factors, indicating confirmed impairment of cognitive functions. In the case of stroke, the elderly illness triggers 31, 32, 33 can comprise a trigger for triggering the occurrence of measuring parameters indicating stroke based on measuring parameters associated with the possibly permanent cognitive or motor impairment and/or indicating the time of an acute stroke episode in the patient dataflow pathway 213, 223, 233.

Another parametric payment can be transferred by triggering measuring parameters in the patient dataflow pathway 213, 223, 233 indicating the initiation of long-term care phase by means of the elderly illness trigger of the core engine 3. This is achieved by triggering 33, in the case of an acute elderly illness 71, . . . , 75, an acute treatment phase of the serious elderly illness 71, . . . , 73 or, in case of a chronic elderly illness 71, . . . , 73, by triggering 33 of a first treatment phase of the chronic elderly illness 71, . . . , 73. For example, acute or first treatment phase parameters indicating surgery and/or chemotherapy and/or radiotherapy and/or reconstructive surgery can be triggered in the patient dataflow pathway 213, 223, 233 via the elderly illness trigger 33 of the core engine 3, For example, the parametric payment can only be transferred by triggering 33 acute or first treatment phase parameters, indicating surgery and/or chemotherapy and/or radiotherapy and/or reconstructive surgery. In the example of dementia, the elderly illness triggers for triggering 33 the first long-term care treatment phase of the chronic elderly illness 71, . . . , 75 can comprise a first treatment phase parameter indicating psychiatric or old-age in--patient care associated with the risk exposure component 21, 22, 23, etc. comprising acute in-patient admission parameters as a result of deterioration in dementia status requiring urgent treatment. In the example of stroke, the elderly illness triggers for triggering 33 the first long-term care treatment phase of the chronic elderly illness 71, . . . , 75 can comprise a first long-term care treatment phase parameter indicating a measured time interval of the risk exposure component 21, 22, 23, etc. spent in the hospital due to the triggered stroke.

Finally, as an alternative, a parametric payment is transferred, in the case of an acute elderly illness 71, . . . , 75, only by triggering 33 recovery phase parameters and/or terminal prognosis parameters and/or an ongoing care or management phase. in case of dementia, the elderly illness triggers 31, . . . , 35 for triggering an ongoing care or management phase of the chronic elderly illness 71, . . . , 73 can comprise ongoing core or management phase parameters indicating permanent cognitive and/or motor impairment requiring continuous supervision by another person and/or ongoing care or management phase parameters indicating permanent cognitive and/or motor impairment requiring constant supervision by another person. In case of stroke, the elderly illness triggers 31, . . . , 35 for triggering an ongoing care or management phase of the chronic elderly illness 71, . . . , 75 can comprise ongoing care or management phase parameters indicating permanent impairments of the cognitive functions and/or permanent cognitive and/or motor impairment requiring continuous supervision by another person and/or permanent cognitive and/or motor impairment requiring constant supervision by another person. For ongoing treatment, the allocated total parametric payment can be determined at least based on the risk-related component data 211, 221, 231 and/or on the likelihood of the risk exposure for one or a plurality of the pooled risk exposure components 21, 22, 23, etc. based on the risk-related component data 211, 221, 231 and wherein a first portion is transferred up to predefined percentage of said total payment sum and the following portions are transferred up to the residual part given by said total payment sum. Such an exemplary payment draw-down, as it can be provided by the resource pooling system 1 in the event of triggering of an elderly illness at a risk exposure component, is illustrated in the diagram of FIG. 1.

As mentioned, the triggers 31, . . . , 35 are uni- or bidirectionally connected with the predefined searchable table 7 of acute or chronic elderly illnesses 71, . . . , 75, wherein the triggering 31, . . . , 35 is performed based on the acute or chronic elderly illness 71, . . . , 75 parameters stored in the predefined searchable table 7. The predefined searchable table 7 is multi-dimensionally structured, for instance as a multidimensional hash table, by means of the multi-layered data structure with predefined searchable acute and/or chronic elderly illness 71, . . . , 75 parameters for triggering by means of elderly illness triggers 31, . . . , 35 in the patient dataflow pathway 213, 223, 233. Each elderly illness 71, . . . , 75 selectable in the multidimensional table has assigned triggerable measuring parameters according to the trigger-step to be performed by means of the multi-pillar trigger 31, . . . , 35 or the resource pooling system 1, i.e., trigger 31 (serious elderly illness) and/or trigger 32 (acute broken bone trauma) and/or trigger 33 (initial long-term care (LTC)) and/or trigger 34 (assistance services) and/or trigger 35 (assisted living benefits). The stored trigger parameters of the multi--pillar trigger 31, . . . , 35 of the predefined searchable table 7 can for example comprise the mentioned trigger dependencies. Furthermore, as an alternative embodiment, the predefined searchable table 7 can also comprise a predefined amount for the various parametric payments assigned to the corresponding trigger 31, . . . , 35. The amount can be fixed for a time period contracted with the risk-exposed component. However, in a preferred alternative embodiment, the parametric payments transferable from the pooled resources via the resource pooling system 1 are dynamically adaptable by the system 1, for example based on the pooled resources or based upon dynamically-checked changing medical conditions or other boundary conditions to the system 1, respectively to the associated and transferred risks.

The predefined searchable acute and/or chronic elderly illness 71, . . . , 75 parameters for triggering by means of elderly illness triggers 31, . . . , 35 in the patient dataflow pathway 213, 223, 233 comprise said predefined values and trigger measuring parameter definition. For example, a heart attack of specified severity can be defined as death of heart muscle, due to inadequate blood supply, that has resulted in all of the following evidence of acute myocardial infarction: Typical clinical symptoms are for example characteristic chest pain, and/or new characteristic in electrocardiographic changes and/or the characteristic rise of cardiac enzymes or troponins recorded at the following levels or higher, wherein troponin T>200 ng/L (0.2 ng/ml or 0.2 ug/L) and troponin I>500 ng/L 10.5 ng/ml or 0.5 ug/L). The triggered evidence must show a definite acute myocardial infarction. As for the above definition, related elderly illnesses are sometimes not covered by the trigger, such as other acute coronary syndromes or angina without myocardial infarction. Such related elderly illnesses can be captured by other correspondingly adapted triggers. Another example is the trigger measuring parameters for stroke—resulting in permanent symptoms. The definition can be given as death of brain tissue due to inadequate blood supply or haemorrhage within the skull resulting in permanent neurological deficiency with persisting clinical symptoms. Again, there are related elderly illnesses which have to be measured and captured by separate triggers, since for the above definition, the following elderly illnesses are not covered: Transient ischaemic attack and/or traumatic injury to brain tissue or blood vessels, and/or death of tissue of the optic nerve or retina/eye stroke. A last example is acute broken bone trauma, In this example, the trigger measuring parameters may simply comprise a listing for measuring different bone fractures, such as closed fracture of the skull, open fracture of the skull, fracture of the vertebra, fracture of the shoulder blade, fracture of the jaw, fracture of the sternum, fracture of the pelvis, fracture of the wrist, fracture of the hand, fracture of the upper leg, fracture of the knee, fracture of the lower leg, fracture of the arm, fracture of the cheekbone, fracture of the foot, fracture of the ankle, fracture of the ribs, and fracture of the collar bone. The trigger parameters of the system 1 may be tuned, so that the system only triggers and transfers the fracture cover benefit to the risk exposed unit 21, 22, 23 for the following fractures as defined in official definition publications, such as ‘Black's Medical Dictionary’ (39th edition): comminuted, complicated, compound, depressed, greenstick, pathological and simple, and the risk-cover transfer will not be performed for any other type of fracture. If more than one of the above fractures occurs at any time, the system may only transfer one parametric fracture cover benefit for one of the fractures, or a plurality of parametric payments. Furthermore, time limits or thresholds can be set for the system 1, where the system 1 only will transfer parametric payments for one fracture suffered during any 12 month period, for instance. The first 12 month period can for example begin on the commencement date, and then each subsequent 12 month period will begin on each anniversary of the commencement date.

Finally, in a further specified alternative embodiment, an independent verification elderly illness trigger of the resource pooling system 1 can be activated in the event of triggering of the occurrence of indicators for elderly illness 71, . . . , 75 in the patient dataflow pathway 213, 223, 233 of a risk exposure component 21, 22, 23, etc. by means of the elderly illness trigger 31, and wherein the independent verification elderly illness trigger is additionally triggering with regard to the occurrence indicators for elderly illness 71, . . . , 75 in an alternative patient dataflow pathway with independent measuring parameters from the primary patient dataflow pathway 213, 223, 233 to verify the occurrence of the elderly illness 71, . . . , 73 at the risk exposure component 21, 22, 23, etc. As an alternative, the parametric draw-down or predefined transfer of payments is only assigned to the corresponding trigger-flag if the occurrence of the elderly illness 71, . . . , 73 at the risk exposure component 21, 22, 23, etc. is verified by the independent verification elderly illness trigger.

1. A resource pooling system for multi-pillar triggered risk transfer associated with prolonged independent living under elderly illness occurrence by providing dynamic self-sufficient risk protection for a variable number of risk exposure components by the resource pooling system, wherein the risk exposure components are connected to the resource pooling system by a plurality of payment receiving devices configured to receive and store payments from the risk exposure components for the pooling of their risks and resources, wherein the resource pooling system comprises a filter device configured to capture age-related parameters of risk exposure components and filter risk exposure components associated with an age-related parameter greater than a predefined age-threshold value by the predefined age-threshold value, and wherein the resource pooling system provides automated risk protection for each of the connected risk exposure components based on received and stored payments and the age-related parameters of the risk exposure components triggering the predefined age-threshold value, the resource pooling system comprising:
processing circuitry configured to
generate a multi-dimensional trigger-table comprising a multi-layered data structure with predefined searchable acute and/or chronic elderly illness parameters for triggering by elderly illness triggers in a patient dataflow pathway, the predefined parameters of a first-layer data structure providing measuring parameters for the occurrence of serious elderly illnesses, the predefined parameters of a second-layer data structure providing measuring parameters for the occurrence of an acute broken bone trauma, the predefined parameters of a third-layer data structure providing measuring parameters for long-term care indications, the predefined parameters of a fourth-layer data structure providing measuring parameters for assisted living indications, and the predefined parameters of a fifth-layer data structure providing measuring parameters for extended assistance indications, the resource pooling system comprising elderly illness triggers triggering measuring values in patient dataflow pathways of the connected risk exposure components, and
when an occurrence of an acute or chronic elderly illness on the patient dataflow pathway of a risk exposure component is triggered by exceeding one of the predefined searchable acute and/or chronic elderly illnesses parameters, set a corresponding trigger-flag and assign a parametric transfer of payments to the corresponding trigger-flag, wherein a loss associated with the acute or chronic elderly illness is distinctly covered by the resource pooling system based on the respective trigger-flag and based on the received and stored payment parameters from risk exposure components by at least one parametric payment transfer from the resource pooling system to the risk exposure component.

2. The resource pooling system according to claim 1, wherein the predefined parameters of the first-layer data structure providing measuring parameters for the occurrence of serious elderly illnesses comprise at least parameters indicating the occurrence of dementia, heart attack, cancer, stroke, coronary artery by-pass surgery, Alzheimer's disease, blindness, deafness, kidney failure., major organ transplant, multiple sclerosis, HIV/AIDS contracted by blood transfusion or during an operation, Parkinson's disease, paralysis of limb or terminal illness in the patient dataflow pathway.

3. The resource pooling system according to claim 1, wherein the predefined parameters of the first-layer data structure providing measuring parameters for the occurrence of serious elderly illnesses comprise at least elderly illness trigger parameters for triggering the occurrence of measuring parameters indicating dementia based on measuring parameters associated with the permanent clinical loss of the ability to remember and/or reason and/or perceive, understand, express and give effect to ideas in the patient dataflow pathway.

4. The resource pooling system according to claim 3, wherein the triggered measuring parameters indicating dementia comprise physical parameters and/or psychological parameters and/or biochemical parameters and/or cognitive factors based on adrenal exhaustion factors and/or food and chemical reaction factors and/or nutritional deficiency factors and/or stress factors and/or depression factors, and/or denial factors, indicating confirmed impairment of cognitive functions.

5. The resource pooling system according to claim 1, wherein the processing circuitry is further configured to process risk-related component data and to provide a likelihood of the risk exposure for one or a plurality of the pooled risk exposure components based on the risk-related component data, wherein receiving and preconditioned storage of payments from risk exposure components for the pooling of their risks is dynamically determinable based on a total risk and/or the likelihood of the risk exposure of the pooled risk exposure components.

6. The resource pooling system according to claim 5, wherein the total risk of the pooled risk exposure components comprises elderly illness risk contributions of each pooled risk exposure component associated with risk exposure in relation to a possibility of triggering at least one acute or chronic elderly illnesses, wherein elderly illness losses occur as a consequence of the triggered diagnosis of risk exposure components with one of the searchable elderly illnesses based upon the predefined trigger measuring values.

7. The resource pooling system according to claim 1, wherein the processing circuitry is further configured to dynamically adapt the elderly illness triggers based on time-correlated incidence data for an elderly illness condition and/or diagnosis or treatment conditions indicating improvements in diagnosis or treatment.

8. The resource pooling system according to claim 1, wherein the processing circuitry is further configured to dynamically adapt the number of pooled risk exposure components to a range where non-covariant occurring risks covered by the resource pooling system affect only a relatively small proportion of the total pooled risk exposure components at a given time.

9. The resource pooling system according to claim 1, wherein the elderly illness triggers trigger elderly illness parameters indicating psychiatric or old-age in--patient care associated with the risk exposure component comprising acute in-patient admission parameters as a result of deterioration in dementia status requiring urgent treatment.

10. The resource pooling system according to claim 1, wherein the elderly illness triggers triggering an ongoing care or management phase of the chronic elderly illness comprise ongoing care or management phase parameters indicating permanent cognitive and/or motor impairment requiring continuous supervision by another person and/or ongoing care or management phase parameters indicating permanent cognitive and/or motor impairment requiring constant supervision by another person.

11. The resource pooling system according to claim 1, wherein the elderly illness triggers comprise on elderly illness trigger for triggering the occurrence of measuring parameters indicating stroke based on measuring parameters associated with possibly permanent cognitive or motor impairment and; or indicating the time of an acute stroke episode in the patient dataflow pathway.

12. The resource pooling system according to claim 1, wherein the elderly illness triggers triggering treatment phase of the chronic elderly illness comprise treatment phase parameters indicating a measured time interval of the risk exposure component spent in the hospital due to the triggered stroke.

13. The resource pooling system according to claim 1, wherein the elderly illness triggers triggering an ongoing care or management phase of the chronic elderly illness comprise ongoing care or management phase parameters indicating permanent impairments of cognitive functions and/or permanent cognitive and/or motor impairment requiring continuous supervision by another person and/or permanent cognitive and/or motor impairment requiring constant supervision by another person.

14. The resource pooling system according to claim 1, wherein the elderly illness triggers further comprise an elderly illness trigger for triggering measuring parameters indicating alcohol and/or drug abuse in the patient dataflow pathway, wherein upon triggering measuring parameters indicating alcohol and/or drug abuse, the processing circuitry rejects the related risk exposure component from pooling of the risk and resources.

15. The resource pooling system according to claim 1, wherein acute treatment phase parameters are triggerable by the elderly illness triggers of acute elderly illness, indicating surgery and/or chemotherapy and/or radiotherapy and/or reconstructive surgery in the patient dataflow pathway,

16. The resource pooling system according to claim 1, wherein a plurality of transferred payment portions are generatable to add up to the allocated total parametric payment and to be transferred with a time shift.

17. The resource pooling system according to claim 16, wherein the allocated total parametric payment is determinable at least based on the risk-related component data and/or on a likelihood of the risk exposure for one or a plurality of the pooled risk exposure components based on the risk--related component data, and wherein a first portion is transferred up to predefined percentage of the total payment sum and the following portions are transferred up to the residual part given by the total payment sum.

18. The resource pooling system according to claim 1, wherein the processing circuitry is further configured to request a periodic payment transfer from the risk exposure components to the resource pooling system by the plurality of payment receiving devices, and interrupt the risk protection for the risk exposure components when the periodic transfer is no longer detectable.

19. The resource pooling system according to claim 18, wherein the processing circuitry interrupts or waives the request for periodic payment transfer when occurrence of indicators for elderly illness is triggered in the patient dataflow pathway of the risk exposure component.

20. The resource pooling system according to claim 1, wherein the processing circuitry is further configured to trigger an independent verification elderly illness trigger, which is activated in the event of triggering of the occurrence of indicators for elderly illness in the patient dataflow pathway of a risk exposure component by the elderly illness triggers, and which additionally is a trigger for the occurrence of indicators for elderly illness in an alternative patient dataflow pathway with independent measuring parameters from the primary patient dataflow pathway to verify the occurrence of the elderly illness at the risk exposure component.

21. The resource pooling system according to claim 20, wherein the parametric transfer of payments is only assigned to the corresponding trigger-flag when the occurrence of the elderly illness at the risk exposure component is verified by the independent verification elderly illness trigger.

22. The resource pooling system according to claim 1, wherein the processing circuitry is further configured to monitor the patient dataflow pathway by capturing a patient measuring parameter of the patient dataflow pathway at least periodically and/or within predefined time frames.

23. The resource pooling system according to claim 1, wherein the processing circuitry is further configured to dynamically monitor the patient dataflow pathway by triggering of patient measuring parameters of the patient dataflow pathway transmitted from associated measuring systems.

24. A method for a patient data-driven system for multi-pillar triggered risk transfer associated with prolonged independent living under elderly illness occurrence by providing dynamic self-sufficient risk protection for a variable number of risk exposure components via resource pooling system, wherein the risk exposure components ore connected to the resource pooling system by a plurality of payment receiving devices configured to receive and store payments from the risk exposure components for the pooling of their risks and resources, wherein the resource pooling system comprises a filter device configured to capture age-related parameters of risk exposure components and filter risk exposure components associated with an age-related parameter greater than a predefined age threshold value via the predefined age-threshold value, and wherein the resource pooling system provides automated risk protection for each of the connected risk exposure components based on received and stored payments and the age-related parameters of the risk exposure components triggering the predefined age-threshold value, the method comprising:
generating, by processing circuitry of the resource pooling system, a multi-dimensional trigger-table comprising a multi-layered data structure with predefined searchable acute and/or chronic elderly illnesses parameters for triggering, by elderly illness triggers in a patient dataflow pathway, the predefined parameters of a first-layer data structure providing measuring parameters for the occurrence of serious elderly illnesses, the predefined parameters of a second-layer data structure providing measuring parameters for the occurrence of an acute broken bone trauma, the predefined parameters of a third-layer data structure providing measuring parameters for long-term care indications, the predefined parameters of a fourth-layer data structure providing measuring parameters for assisted living indications, and the predefined parameters of a fifth-layer data structure providing measuring parameters for extended assistance indications, the resource pooling system comprising elderly illness triggers triggering measuring values in patient dataflow pathways of the connected risk exposure components; and
when an acute or chronic elderly illness occurs on the patient dataflow pathway of a risk exposure component or is triggered by exceeding one of the predefined searchable acute and/or chronic elderly illness parameters, setting a corresponding trigger--flag via the resource pooling system, and assigning a parametric transfer of payments to the corresponding trigger-flag, wherein a loss associated with the acute or chronic elderly illness is covered separately by the resource pooling system based on the respective trigger-flag and based on the received and stored payment parameters from risk exposure components by at least one parametric payment transfer from the resource pooling system to the risk exposure component.

25. The method according to claim 24, wherein the predefined parameters of the first-layer data structure providing measuring parameters for the occurrence of serious elderly illnesses comprise at least parameters indicating the occurrence of dementia, heart attack, cancer, stroke, coronary artery by-pass surgery, Alzheimer's disease, blindness, deafness, kidney failure., major organ transplant, multiple sclerosis, HIV/AIDS contracted by blood transfusion or during an operation, Parkinson's disease. paralysis of limb or terminal illness in the patient dataflow pathway.

26. The method according to claim 24, wherein the predefined parameters of the first-layer data structure providing measuring parameters for the occurrence of serious elderly illnesses comprise at least elderly illness trigger parameters for triggering the occurrence of measuring parameters indicating dementia based on measuring parameters associated with the permanent clinical loss of the ability to remember and/or reason and/or perceive, understand, express and give effect to ideas in the patient dataflow pathway.

27. The method according to claim 26, wherein the measuring parameters indicating dementia comprise physical parameters and/or psychological parameters and/or biochemical parameters and/or cognitive factors based on adrenal exhaustion factors and/or food and chemical reaction factors and/or nutritional deficiency factors and/or stress factors and/or depression factors, or denial factors, indicating confirmed impairment of cognitive functions.

28. The method according to claim 24, further comprising:
processing, by the processing circuitry of the resource pooling system, risk-related component data and providing a likelihood of the risk exposure for one or a plurality of the pooled risk exposure components based on the risk-related component data, wherein receiving and preconditioned storage of payments from risk exposure components for the pooling of their risks is dynamically determinable based on a total risk and/or the likelihood of the risk exposure of the pooled risk exposure components.

29. The method according to claim 28, wherein the total risk of the pooled risk exposure components comprises elderly illness risk contributions of each pooled risk exposure components associated with risk exposure in relation to a possibility of triggering of at least one acute or chronic elderly illnesses, wherein elderly illness losses occur as a consequence of the triggered diagnosis of risk exposure components with one of the searchable elderly illnesses based upon the predefined trigger measuring values.

30. The method according to claim 24, further comprising:
dynamically adapting the elderly illness triggers based on time-correlated incidence data for an elderly illness condition and/or diagnosis or treatment conditions indicating improvements in diagnosis or treatment.

31. The method according to claim 24, further comprising:
dynamically adapting the number of pooled risk exposure components to a range where non-covariant occurring risks covered by the resource pooling system affect only a relatively small proportion of the total pooled risk exposure components at a given time,

32. The method according to claim 24, wherein the elderly illness triggers trigger elderly illness parameters indicating psychiatric or old-age in-patient care associated with the risk exposure component comprising acute in-patient admission parameters as a result of deterioration in dementia status requiring urgent treatment.

33. The method according to claim 24, wherein the elderly illness triggers trigger an ongoing care or management phase of the chronic elderly illness comprise ongoing care or management phase parameters indicating permanent cognitive and/or motor impairment requiring continuous supervision by another person and/or ongoing care or management phase parameters indicating permanent cognitive and/or motor impairment requiring constant supervision by another person.

34. The method according to claim 24, wherein an elderly illness trigger of the elderly illness triggers triggers the occurrence of measuring parameters indicating a stroke based on measuring parameters associated with the possibly permanent cognitive or motor impairment and/or indicating the time of an acute stroke episode in the patient dataflow pathway.

35. The method according to claim 34, wherein the elderly illness triggers triggering a treatment phase of the chronic elderly illness comprise treatment phase parameters indicating a measured time interval of the risk exposure component spent in a hospital due to the triggered stroke.

36. The method according to claim 24, wherein the elderly illness triggers triggering an ongoing care or management phase of the chronic elderly illness comprise ongoing care or management phase parameters indicating permanent impairments of cognitive functions and/or permanent cognitive and/or motor impairment requiring continuous supervision by another person and/or permanent cognitive and/or motor impairment requiring constant supervision by another person. 15

37. The method according to claim 24, wherein an elderly illness trigger of the elderly illness triggers further triggers measuring parameters indicating alcohol and/or drug abuse in the patient dataflow pathway, the method further comprising:
upon triggering measuring parameters indicating alcohol and/or drug abuse, rejecting the related risk exposure component from pooling of the risk and resources.

38. The method according to claim 24, wherein the elderly illness triggers of acute elderly illness trigger acute treatment phase parameters indicating surgery and/or chemotherapy and/or radiotherapy and/or reconstructive surgery in the patient dataflow pathway.

39. The method according to claim 24, further comprising:
generating a plurality of transferred payment portions to odd up to the allocated total parametric payment and to be transferred with a time shift.

40. The method according to claim 39, further comprising:
determining the allocated total parametric payment at least based on the risk-related component data and/or a likelihood of the risk exposure one or a plurality of the pooled risk exposure components based on the risk-related component data, and wherein a first portion is transferred up to predefined percentage of the total payment sum and the following portions are transferred up to the residual t given by the total payment sum.

41. The method according to claim 24, further comprising:
requesting a periodic payment transfer from the risk exposure components to the resource pooling system by the plurality of payment receiving devices: and
interrupting the risk protection for the risk exposure components when the periodic transfer is no longer detectable.

42. The method according to claim 41, further comprising:
interrupting or waiving the request for periodic payment transfer when the occurrence of indicators for elderly illness is triggered in a patient dataflow pathway of a risk exposure component.

43. The method according to claim 24, further comprising:
activating on independent verification elderly illness trigger of the resource pooling system in the event of triggering of the occurrence of indicators for elderly illness in the patient dataflow pathway of a risk exposure component by the elderly illness triggers, and which is additionally a triggering for the occurrence of indicators for elderly illness in an alternative patient dataflow pathway with independent measuring parameters from the primary patient dataflow pathway to verify the occurrence of the elderly illness at the risk exposure component.

44. The method according to claim 43, wherein the parametric: transfer of payments is only assigned to the corresponding trigger-flag when the occurrence of the elderly illness at the risk exposure component is verified by the independent verification elderly illness trigger.

45. The method according to claim 24, further comprising:
monitoring the patient dataflow pathway by capturing patient measuring parameters of the patient dataflow pathway at least periodically and/or within predefined time frames.

46. The method according to claim 24, further comprising:
dynamically monitoring the patient dataflow pathway by triggering of patient measuring parameters of the patient dataflow pathway transmitted from associated measuring systems.
